(19) United States
(12) Patent Application Publication
(10) Pub. No.: US 2008/0311234 A1
(43) Pub. Date: Dec. 18, 2008

(54) Oil-in-Water Emulsified Composition, and External Preparation for Skin and Cosmetics Using the Composition

(76) Inventors: Tadashi Yoneda, Chiba (JP); Naoko Ito, Chiba (JP); Kazuo Furuya, Chiba (JP)

Correspondence Address: SUGHRUE MION, PLLC, 2100 PENNSYLVANIA AVENUE, N.W., SUITE 800, WASHINGTON, DC, 20037, US

(21) Appl. No.: 10/593,692

(22) PCT Filed: Mar. 22, 2005
(86) PCT No.: PCT/JP05/05641

§ 371 Date: Sep. 20, 2006

Related U.S. Application Data
(60) Provisional application No. 60/558,548, filed on Apr. 2, 2004.

(30) Foreign Application Priority Data
Mar. 24, 2004 (JP) 2004-86669

(51) Int. Cl. A61K 35/66; A61Q 19/00

(52) U.S. Cl. 424/780

(57) ABSTRACT

An oil-in-water emulsified composition suitable for external preparation for skin and cosmetic product, which comprised 0.1 to 5% by mass of (A) lipopeptide compound derived from a microorganism such as surfactin and its analogous compound, 0.05 to 1.5% by mass of (B) xanthan gum, 25 to 70% by mass of (C) oil component and (D) water, free from non-ionic surfactants and acrylic water-soluble polymers, and is excellent in feeling upon use, moisture retention, emollient property and stability as well as environmental suitability and safety for living organisms, and external preparation for skin and cosmetic product using the composition.

### CROSS-REFERENCE TO THE RELATED APPLICATIONS

This is an application filed pursuant to 35 U.S.C. Section 111(a) with claiming the benefit of U.S. Provisional application Ser. No. 60/558,548 filed Apr. 2, 2004, under the provision of 35 U.S.C. Section 111(b), pursuant to 35 U.S.C. Section 119(e)(1).

### TECHNICAL FIELD

The present invention relates to an oil-in-water emulsified composition. Specifically, the present invention relates to an oil-in-water emulsified composition comprising lipopeptide compounds derived from microorganisms and xanthan gum, which is excellent in feeling upon use, moisture retention, emollient property and stability as well as environmental suitability and safety for living organisms.

### BACKGROUND ART

Oil-in-water emulsified compositions, providing fresh feeling upon use, are being widely used in cosmetics, quasi-drugs and the like.

Generally, emulsified products are unstable to heat, and various methods for stably retaining the emulsified state are known. Among them, a method of increasing viscosity of the external phase is often employed. In case of oil-in-water emulsified composition, natural water-soluble polymers such as xanthan gum, locust bean gum, guar gum and carrageenan, and synthetic water-soluble polymers such as polyvinylalcohol, polyvinylpyrrolidone, sodium polyacrylate, carboxyvinyl polymer, alkyl-modified carboxyvinyl polymer and copolymer of alkyl-modified acrylic acid/methacrylic acid are used for the purpose of increasing the viscosity of the external phase.

As surfactants used in oil-in-water emulsified compositions, nonionic surfactants such as sorbitan fatty acid ester, polyoxyethylene sorbitan fatty acid ester and polyoxyethylene alkylether have been conventionally used.

However, with respect to emulsified compound used in external preparation for skin and cosmetics, due to increasing concern in not only safety and mildness for skin but also environmental suitability, it is required to use a material having as high safety for the living body and as high environmental suitability as possible, and reduction in usage of synthetic water-soluble polymers and nonionic surfactants which include petroleum in their starting materials is being demanded.

In order to solve these problems, various studies are being made on oil-in-water emulsified compositions using water-soluble polymers and surfactants derived from natural materials. External preparation for skin and cosmetics using lipopeptide compounds derived from microorganisms are disclosed, for example, in JP-A-2000-327591 (WO99/62482), JP-A-2003-176211(WO03/013446), JP-A-2003-95853, JP-A-2003-12445, JP-A-2003-277220 and JP-A-2003-277250. However, in those techniques, there remain problems that emulsification is insufficient or that a stable emulsified product cannot be obtained.

### DISCLOSURE OF THE INVENTION

An object of the present invention lies in provision of an oil-in-water emulsified composition which is excellent in feeling upon use, moisture retention, emollient property and stability as well as environmental suitability and safety for living organisms.

Another object of the present invention lies in provision of external preparations for skin and cosmetics using the oil-in-water emulsified composition.

As a result of intensive investigations to solve this problem, the present inventors have found that by using lipopeptide compounds derived from microorganisms and xanthan gum in combination, even without using synthetic water soluble polymer or nonionic surfactant, good emulsification can be attained to thereby obtain an oil-in-water emulsified composition which is excellent in feeling upon use, moisture retention, emollient property and stability as well as environmental suitability and safety for living organisms, and thus completed the present invention.

Accordingly, the invention relates to the following items.

1. An oil-in-water emulsified composition, which comprises 0.1 to 5% by mass of (A) lipopeptide compound derived from a microorganism, 0.05 to 1.5% by mass of (B) xanthan gum, (C) oil component and (D) water.
2. The oil-in-water emulsified composition according to the above item 1, wherein the content of the oil component is from 25 to 70% by mass.
3. The oil-in-water emulsified composition according to the above item 1, wherein the water content is from 15 to 55% by mass.
4. The oil-in-water emulsified composition according to the above item 1, wherein the microorganism-derived lipopeptide compound (A) is at least one species selected from surfactins, its analogous compounds and salts thereof.
5. The oil-in-water emulsified composition according to the above item 4, wherein the surfactin or its analogous compound comprises at least one or more compounds as represented by the formula (1) below:

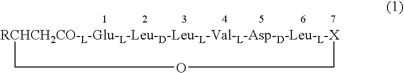

(in the formula, X represents an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine, and R represents a normal alkyl group having from 8 to 14 carbon atoms, an isoalkyl group having from 8 to 14 carbon atoms or an anteisoalkyl group having from 8 to 14 carbon atoms).
6. The oil-in-water emulsified composition according to the above item 5, wherein X is residue of leucine, isoleucine or valine.
7. The oil-in-water emulsified composition according to the above item 4, wherein the salt is at least one compound selected from the group consisting of sodium salt, potassium salt, monoethanolamine salt, diethanolamine salt, triethanolamine salt, arginine salt and lysine salt.
8. The oil-in-water emulsified composition according to the above item 4, wherein the microorganism-derived lipopeptide compound (A) is sodium surfactin.
9. The oil-in-water emulsified composition according to any one of the above items 1 to 8, comprising no nonionic surfactant.
10. The oil-in-water emulsified composition according to any one of the above items 1 to 9, comprising no acrylic acid-based water-soluble polymer.
11. An external preparation for skin comprising the oil-in-water emulsified composition according to any one of the above items 1 to 10.
12. A cosmetic comprising the oil-in-water emulsified composition according to any one of the above items 1 to 10.

The invention is explained below in detail.

Examples of the lipopeptide compound (A) used in the invention include lipopeptide compounds produced by microorganisms of genus Bacillus such as Bacillus subtilis described in JP-A-2000-327591 (WO99/62482). Preferable examples include salts of surfactin and salts of analogous compounds thereof.

The surfactin herein refers to a compound represented by the formula (1):

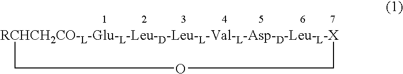

or a composition containing two or more kinds of the compounds represented by the formula (1).

In the above formula (1), X represents an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine. Preferred X is leucine residue, isoleucine residue or valine residue.

R is a normal alkyl group having 8 to 14 carbon atoms, an isoalkyl group having 8 to 14 carbon atoms or an anteiso-alkyl group having 8 to 14 carbon atoms. The normal alkyl group is a straight chain alkyl group; the isoalkyl group usually has a structure which comprises (CH₃)₂CH—(CH₂)ₙ—; and the anteiso-isoalkyl group usually has a structure which comprises CH₃—CH₂—CH(CH₃)— (CH₂)ₙ—.

The analogous compound of surfactin refers to compounds having amino acid(s) substituted by other amino acid (s) in the aforementioned formula (1). Specifically, examples of such a compound include compounds where L-leucine as the second amino acid, L-valine as the fourth amino acid and/or D-leucine as the sixth amino acid are substituted by other amino acid(s), but not limited thereto. Hereinafter, “surfactin or an analogous compound thereof” may be referred to as “surfactin”.

Surfactin can be utilized as the inorganic salt or the organic salt of a compound as represented by the above formula (1). Metal used for counter ion may be of any kind, for example, alkali metals such as sodium, potassium and lithium and alkaline earth metals such as calcium and magnesium, as long as the metal forms a salt with surfactin.

Examples of the organic salt include salts of trimethylamine, triethylamine, tributylamine, monoethanolamine, diethanolamine, triethanolamine, lysine, arginine and choline.

Among these, salts of sodium, potassium, monoethanolamine, diethanolamine, triethanolamine, lysine and arginine are preferred, and sodium salt is particularly preferred.

As sodium surfactin, it is preferred to use a product available on the market under the trade name of Aminofect (registered trademark of SHOWA DENKO K.K.).

The amount of lipopeptide compound contained in the composition of the present invention is preferably 0.1 to 5% by mass, more preferably 0.5 to 4% by mass, and still more preferably 0.8 to 3% by mass. When the amount is less than 0.1% by mass, the stability of the composition may be insufficient, and also in cases where the compound is used in an amount exceeding 5% by mass, it does not lead to increase in effects which is commensurate with the excess amount used can be obtained.

External preparations for skin and cosmetics using microorganism-derived lipopeptide compounds are disclosed, for example, in JP-A-2000-327591 (WO99/62482), JP-A-2003-176211(WO03/013446), JP-A-2003-95853, JP-A-2003-12445, JP-A-2003-277220 and JP-A-2003-277250 mentioned in the “background art” paragraph. However, none of the documents describes that by using a microorganism-derived lipopeptide compound in combination with xanthan gum, good emulsification property can be obtained without using synthetic water-soluble polymer or nonionic surfactant, and that thereby an oil-in-water emulsified composition excellent in feeling upon use, moisture retention, emollient property and stability as well as environmental suitability and safety for living organisms can be obtained. The present invention is the first to disclose the technique.

The xanthan gum (B) used in the present invention is not particularly limited and any kind can be used as long as the xanthan gum is generally used as raw material for external preparation for skin. Preferable examples of the xanthan gum usable in the present invention include Echo Gum, Echo Gum T and Echo Gum BT distributed by DAINIPPON PHARMACEUTICAL CO., LTD. The compounding amount of the xanthan gum in the composition of the present invention is preferably 0.05 to 1.5% by mass, more preferably 0.08 to 0.7% by mass, still more preferably 0.1 to 0.4% by mass. If the amount of the xanthan gum is less than 0.05% by mass, sufficient stability of the emulsified composition cannot be obtained. The amount of the xanthan gum exceeding 1.5% by mass is unpreferable, since it would deteriorate feeling upon using the composition of the present invention.

The oil-in-water emulsified composition of the present invention contains oil component (C). Any oil material can be employed unless it is arbitrarily mixed with water. It is preferable that one or more selected from hydrocarbons, natural fats and oils, fatty acids, higher alcohols, alkyl glyceryl ethers, esters and silicone oils be compounded in. The total compounding amount of oil components is preferably 25 to 70% by mass, more preferably 30 to 60% by mass, based on the total amount of the composition.

The oil-in-water emulsified composition of the present invention contains water (D). The water content is to be contained as balance, and a preferable range of the water content is from 15 to 55% by mass, and particularly preferred is from 20 to 50% by mass.

The oil-in-water emulsified composition of the present invention does not necessarily require use of nonionic surfactants or acrylic acid-base water-soluble polymers which are usually employed in production of conventional oil-in-water emulsified compositions and the composition of the present invention substantially does not have to contain such components. That is, preparation of the oil-in-water emulsified composition of the present invention may include addition of nonionic surfactants or acrylic acid base water-soluble polymers or may dispense with such components.

Examples of nonionic surfactant referred to herein include sorbitan fatty acid ester, polyoxyethylene sorbitan fatty acid ester, polyoxyethylene alkyl ether, glycerine fatty acid ester and polyoxyethylene glycerine fatty acid ester.

To be more specific, nonionic surfactants such as polyoxyethylene (10) alkyl (12,13) ether, polyoxyethylene lauryl ether, polyoxyethylene cetyl ether, polyoxyethylene stearyl ether,

polyoxyethylene oleyl ether, polyoxyethylene (3,7,12) alkyl (12 to 14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2,10,20) isostearyl ether, polyoxyethyleneoleylcetyl ether, polyoxyethylene (20) arachyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1,2,4,8) cetyl ether, polyoxyethylene (5) polyoxypropylene (1,2,4,8) cetyl ether, polyoxyethylene (10) polyoxypropylene (1,2,4,8) cetyl ether, polyoxyethylene (20) polyoxypropylene (1,2,4,8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifying ethylene glycol monostearate, diethylene glycol laurate, polyethylene glycol myristate, polyethylene glycol palmitate, diethylene glycol stearate, self-emulsifying polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitantristearate, polyoxyethylene (6) sorbitanmonooleate, polyoxyethylene (20) sorbitanmonooleate, polyoxyethylene (20) sorbitan trioleate,
polyoxyethylene (20) coconut oil fatty acid sorbitan, polyoxyethylene (10 to 80) sorbitan monolaurate, polyoxyethylene sorbitan tristearate, polyoxyethylene (20) sorbitan isostearate,
polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil,
polyoxyethylene (60) hydrogenated castor oil, lipophilic glycerin monostearate, lipophilic glycerin monooleate, self-emulsifying glycerin monostearate, coconut oil fatty acid glyceryl, glycerin laurate, glyceryl myristate, glyceryl isostearate, glyceryl ricinoleate, glyceryl monohydroxystearate, glycerin oleate, glyceryl linoleate, glyceryl erucate, glyceryl behenate, wheat germ oil fatty acid glyceride, safflower oil fatty acid glyceryl, hydrogenated soybean fatty acid glyceryl, saturated fatty acid glyceride,
cotton seed oil fatty acid glyceryl, monomyristate glyceryl monoisostearate, beef tallow monoglyceride, monolanolin fatty acid glyceryl, glyceryl sesquioleate, glyceryl distearate,
glyceryl diisostearate, glyceryl diarachidate, sorbitan monolaurate, sorbitan monopalmitate, sorbitan monostearate,
sorbitan monoisostearate, sorbitan monooleate, sorbitan sesquistearate, sorbitan sesquioleate, sorbitantristearate, sorbitan trioleate, coconut oil fatty acid sorbitan, sorbitan isostearate, sorbitan sesquiisostearate, sorbitan distearate,
diglyceryl isopalmitate, poly(4 to 10)glyceryl monolaurate, poly(10)glyceryl monomyristate, poly(2 to 10)glyceryl monostearate, poly(2 to 10)glyceryl monoisostearate, poly(2 to 10)glyceryl monooleate, diglyceryl sesquioleate, poly(2 to 10)glyceryl diisostearate, poly(6 to 10)glyceryl distearate,
diglyceryl triisostearate, poly(10)glyceryl tristearate, poly(10)glyceryl trioleate, poly(2)glyceryl tetraisostearate,
decaglyceryl pentastearate, poly(6 to 10)glyceryl pentaoleate,
poly(10)glyceryl heptastearate, decaglyceryl decastearate, poly(10)glyceryl decaoleate, concentrated poly(6)glyceryl ricinoleate, sucrose fatty acid ester, coconut oil fatty acid sucrose ester, alkyl glucoside, coconut oil alkyl dimethylamine oxide, lauryl dimethylamine oxide, dihydroxyethyl lauryl dimethylamine oxide, stearyl dimethylamine oxide, oleyl dimethylamine oxide, polyoxyethylene coconut oil alkyl dimethylamine oxide, polyoxyethylene (3) glyceryl triisostearate, polyoxyethylene (5) glyceryl triisostearate, polyoxyethylene (10) glyceryl triisostearate, polyoxyethylene (20) glyceryl triisostearate, polyoxyethylene (30) glyceryl triisostearate,
polyoxyethylene (40) glyceryl triisostearate, polyoxyethylene (50) glyceryl triisostearate, polyoxyethylene (60) glyceryl triisostearate, polyoxyethylene (3) glyceryl isostearate, polyoxyethylene (5) glyceryl isostearate, polyoxyethylene (6) glyceryl isostearate, polyoxyethylene (8) glyceryl isostearate,
polyoxyethylene (10) glyceryl isostearate, polyoxyethylene (15) glyceryl isostearate, polyoxyethylene (20) glyceryl isostearate,
polyoxyethylene (25) glyceryl isostearate, polyoxyethylene (30) glyceryl isostearate, polyoxyethylene (40) glyceryl isostearate,
polyoxyethylene (50) glyceryl isostearate, polyoxyethylene (60) glyceryl isostearate, polyoxyethylene (3) glyceryl tristearate,
polyoxyethylene (4) glyceryl tristearate, polyoxyethylene (5) glyceryl tristearate, polyoxyethylene (6) glyceryl tristearate,
polyoxyethylene (10) glyceryl tristearate, polyoxyethylene (20) glyceryl tristearate, polyoxyethylene (4) glyceryl distearate,
polyoxyethylene (3) glyceryl trioleate, polyoxyethylene (5) glyceryl trioleate, polyoxyethylene (10) glyceryl trioleate, polyoxyethylene (20) glyceryl trioleate, polyoxyethylene (30) glyceryl trioleate, polyoxyethylene (40) glyceryl trioleate,
polyoxyethylene (50) glyceryl trioleate, polyoxyethylene (60) glyceryl trioleate, polyoxyethylene sorbit monolaurate, polyoxyethylene (40) sorbit oleate, polyoxyethylene (4) sorbit tetraoleate, polyoxyethylene (3) sorbit tristearate, polyoxyethylene (30) sorbit tetraoleate, polyoxyethylene (40) sorbit tetraoleate, polyoxyethylene (60) sorbit tetraoleate, polyoxyethylene (3) sorbit isostearate, polyoxyethylene (40) sorbit oleate, polyoxyethylene (60) sorbit tetrastearate, polyoxyethylene (6) sorbit hexaoleate, polyoxyethylene sorbit hexastearate and polyoxyethylene (40) sorbit pentaoleate.

Further, examples of the acrylic acid base water-soluble polymer include sodium polyacrylate, carboxyvinyl polymer, alkyl-modified carboxyvinyl polymer, acrylate/methacrylate copolymer, ethylene/acrylic acid copolymer, acrylate/methacrylate alkyl (C 10 to 30) copolymer, acrylic acid based anion polymer and methaacrylic acid based anion polymer.

In the emulsified composition of the present invention, other ingredients which are usually used in conventional external preparations for skin and cosmetics may be optionally compounded in within a range where the present invention can attain the objects of the invention.

Examples of such ingredients include hydrocarbons such as ozokerite, α-olefin oligomer, light isoparaffin, light liquid isoparaffin, squalene, squalane, synthetic squalane, phytosqualane, ceresin, paraffin, polyethylene powder, polybutene, microcrystalline wax, liquid isoparaffin, liquid paraffin, mineral oil and vaseline;

natural waxes such as jojoba oil, carnaubawax, candelilla wax, rice bran wax, shellac, lanolin, mink sebaceous wax, spermaceti wax, sugarcane wax, sperm whale oil, beeswax and montan wax, natural fats and fatty oils such as avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cotton seed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia japonica oil, Camellia kissi oil, cacao butter, Japan wax, beef bone tallow, nest's-foot oil, swine tallow, equine tallow, ovine tallow, shea butter, macadamia nut oil and meadowfoam seed oil;

fatty acids such as lauric acid, myristic acid, palmitic acid, stearic acid, behenic acid, oleic acid, linoleic acid, linolenic acid, γ-linolenic acid, isostearic acid, 12-hydroxystearic acid, undecylenic acid and coconut oil fatty acid;

higher alcohols such as isostearyl alcohol, octyl dodecanol, hexyl decanol, cholesterol, phytosterol, lauryl alcohol, myristyl alcohol, cetyl alcohol, stearyl alcohol, oleyl alcohol, behenyl alcohol and cetostearyl alcohol;

alkylglyceryl ethers such as batyl alcohol, chimyl alcohol, serachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearylpalmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, propylene glycol dicaprate, propylene glycol dioleate, neopentyl glycol dicaprate, neopentyl glycol dioctanoate, glyceryl tricaprylate, glyceryl tri 2-ethyl hexanoate, glyceryl tricaprylate/tricaprate, glyceryl tricaprylate/tricaprate/tristearate, glyceryl triundecylate, glyceryl triisopalmitate, glyceryl triisostearate, trimethylolpropane tri 2-ethylhexanoate, trimethylolpropane triisostearate, pentaerythrityl tetra 2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di 2-ethylhexyl succinate, diisopropyl adipate, diisobutyl adipate, dioctyl adipate, diheptylundecyl adipate, sebacate diethyl, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methyl hydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethylsiloxane-methyl(polyoxyethylene)siloxane-methyl(polyoxypropylene)siloxane copolymer, dimethylsiloxane-methyl (polyoxyethylene)siloxane copolymer, dimethylsiloxane-methyl (polyoxypropylene)siloxane copolymer, dimethylsiloxane-methylcetyl oxysiloxane copolymer, dimethylsiloxane-methyl stearoxysiloxane copolymer, polyether modified silicone, alcohol modified silicone, alkyl modified silicone and amino modified silicone;

polyhydric alcohols such as ethylene glycol, diethylene glycol, triethylene glycol, polyethylene glycol, propylene glycol, dipropylene glycol, polypropylene glycol, glycerin, diglycerin, polyglycerin, 3-methyl-1,3-butanediol, 1,3-butanediol, 1,2-pentanediol and 1,2-hexanediol;

saccharides such as mannitol, sorbitol, xylitol, maltitol, erythritol, pentaerythritol, glucose, sucrose, fructose, lactose, maltose, xylose and trehalose;

polymers such as sodium alginate, carrageenan, agar, furcellaran, guar gum and quince seed, Amorphophalus konjak (arum root) mannan, tamarind gum, tara gum, dextrin, starch, locust bean gum, gum arabic, gum gatti, karaya gum, gum tragacanth, arabinogalactan, pectin, quince, chitosan, curdlan, xanthan gum, gellan gum, cyclodextrin, dextran, pullulan, microcrystalline cellulose, methyl cellulose, ethylcellulose, hydroxyethyl cellulose, hydroxypropyl cellulose, hydroxypropylmethyl cellulose, carboxymethyl cellulose, carboxy starch, cationized cellulose, starch phosphate ester, cationized guar gum, carboxyethyl-hydroxypropylated guar gum, hydroxypropylated guar gum, albumin, casein, gelatin, polyacrylic amide, polyethylene imine, highly polymerized polyethylene glycol, polyvinyl alcohol, polyvinylpyrrolidone, polyvinyl ether, polyacryl amide, acrylic acid copolymer, methacrylic acid copolymer, maleic acid copolymer, vinylpyridine copolymer, vinyl pyrrolidone based polymer, vinyl alcohol/vinyl pyrrolidone copolymer, nitrogen-substituted acrylamide based polymer, amino modified silicone, cationized polymer, dimethylacryl ammonium based polymer, modified silicone and polyoxyethylene/polyoxypropylene copolymer;

alcohols such as ethanol, isopropyl alcohol, 1-butanol, 2-butanol and benzyl alcohol;

anionic surfactants such as coconut oil fatty acid potassium, coconut oil fatty acid sodium, coconut oil fatty acid triethanolamine, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene lauryl ether acetate, sodium polyoxyethylene lauryl ether acetate, polyoxyethylene tridecyl ether acetate, sodium polyoxyethylene tridecyl ether acetate, sodium stearoyl lactate, sodium isostearoyl lactate, sodium lauroyl sarcosine, coconut oil fatty acid sarcosine, sodium coconut oil fatty acid sarcosine, coconut oil fatty acid sarcosine triethanolamine, lauroyl sarcosine, potassium lauroyl sarcosine, lauroyl sarcosine triethanolamine, oleoyl sarcosine, sodium myristoyl sarcosine, sodium stearoyl glutamate, coconut oil fatty acid acyl glutamic acid, potassium coconut oil fatty acid acyl glutamate, sodium coconut oil fatty acid acyl glutamate, coconut oil fatty acid acyl glutamate triethanolamine, lauroylacyl glutamic acid, potassium lauroylacyl glutamate, sodium lauroylacyl glutamate, lauroylacyl glutamate triethanolamine, myristoylacyl glutamic acid, potassium myristoylacyl glutamate, sodium myristoylacyl glutamate, stearoylacyl glutamic acid, potassium stearoylacyl glutamate, disodium stearoylacyl glutamate, sodium hydrogenated beef tallow fatty acid acyl glutamate, sodium coconut oil fatty acid/hydrogenated beef tallow fatty acid acyl glutamate, sodium coconut oil fatty acid methylalanine, lauroyl methylalanine, sodium lauroyl methylalanine, lauroyl methylalanine triethanolamine, sodium myristoyl methylalanine, sodium lauroyl methyltaurine, potassium coconut oil fatty acid methyltaurine, sodium coconut oil fatty acid methyltaurine, magnesium coconut oil fatty acid methyltaurine, sodium myristoyl methyltaurine, sodium palmitoyl methyltaurine, sodium stearoyl methyltaurine, sodium oleoyl methyltaurine, sodium alkane sulfonate, sodium tetradecene sulfonate, sodium sulfosuccinate dioctyl, disodium lauryl sulfosuccinate, sodium coconut oil fatty acid ethyl ester sulfonate, sodium lauryl sulfate, triethanolamine lauryl sulfate, sodium cetyl sulfate, triethanolamine alkyl (11,13,15) sulfate, sodium alkyl (12,13) sulfate, triethanolamine alkyl (12,13) sulfate, alkyl (12,14,16) ammonium sulfate, diethanolamine alkyl (12 to 13) sulfate, triethanolaminealkyl (12 to 14) sulfate, triethanolamine alkyl (12 to 15) sulfate, magnesium coconut oil alkyl sulfate/triethanolamine, lauryl ammonium sulfate, potassium lauryl sulfate, magnesium lauryl sulfate, monoethanolamine lauryl sulfate, diethanolamine lauryl sulfate, sodium myristyl sulfate, sodium stearylsulfate, sodium oleyl sulfate, triethanolamine oleyl sulfate, sodium polyoxyethylene lauryl ether sulfate, triethanolamine polyoxyethylene lauryl ether sulfate, sodium polyoxyethylene (1) alkyl (11,13,15) ether sulfate, triethanolamine polyoxyethylene (1) alkyl (11,13,15) ethersulfate, sodium polyoxyethylene (3) alkyl (11 to 15) ether sulfate, sodium polyoxyethylene (2)alkyl (12,13) ethersulfate, sodium polyoxyethylene (3) alkyl (12 to 14) ethersulfate, sodium polyoxyethylene (3) alkyl (12 to 15) ether sulfate, sodium polyoxyethylene (2) lauryl ether sulfate, sodium polyoxyethylene (3) myristyl ether sulfate, sodium higher fatty acid alkanol amide sulfate ester, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphate, sodium polyoxyethylene alkylphenyl ether phosphate, triethanolamine polyoxyethylene alkylphenyl ether phosphate, polyoxyethylene octyl ether phosphate, polyoxyethylene (10) alkyl (12,13) ether phosphate, polyoxyethylene alkyl (12 to 15) ether phosphate, polyoxyethylene alkyl (12 to 16) ether phosphate, triethanolamine polyoxyethylene lauryl ether phosphate and diethanolamine polyoxyethylene oleyl ether phosphate;

cationic surfactants such as dioctylamine, dimethylstearylamine, trilaurylamine, diethylaminoethylamide stearate, lauryl trimethylammonium chloride, cetyl trimethylammonium chloride, cetyl trimethylammonium bromide, cetyl trimethylammonium saccharin, stearyl trimethylammonium chloride, alkyl (20 to 22) trimethylammonium chloride, lauryl trimethylammonium bromide, alkyl (16,18) trimethylammonium chloride, stearyl trimethylammonium bromide, stearyl trimethylammonium saccharin, alkyl (28) trimethylammonium chloride, di(polyoxyethylene) oleyl methylammonium (2EO) chloride, dipolyoxyethylene stearyl methylammonium chloride, polyoxyethylene (1) polyoxypropylene (25) diethylmethylammonium chloride, tri(polyoxyethylene) stearyl ammonium (5EO) chloride, distearyl dimethylammonium chloride, dialkyl (12 to 15) dimethylammonium chloride, dialkyl (12 to 18) dimethylammonium chloride, dialkyl (14 to 18) dimethylammonium chloride, dicocoyl dimethylammonium chloride, dicetyl dimethylammonium chloride, isostearyllauryl dimethylammonium chloride, benzalkonium chloride, myristyl dimethylbenzyl ammonium chloride, lauryl dimethyl(ethylbenzyl)ammonium chloride, stearyl dimethylbenzyl ammonium chloride, lauryl pyridinium chloride, cetyl pyridinium chloride, lauroyl cholamino formylmethyl pyridinium chloride, stearoyl cholamino formylmethyl pyridinium chloride, alkyl isoquinolinium bromide, methyl benzethonium chloride and benzethonium chloride;

ampholytic surfactants such as 2-alkyl-N-carboxymethyl-N-hydroxyethyl imidazolium betaine, alkyldiamino ethyl glycine hydrochloride, sodium lauryldiamino ethyl glycine, sodium undecyl hydroxyethyl imidazolium betaine, undecyl-N-carboxymethyl imidazolium betaine, disodium coconut oil fatty acid acyl-N-carboxyethyl-N-hydroxyethyl ethylenediamine, disodium coconut oil fatty acid acyl-N-carboxyethoxyethyl-N-carboxyethyl ethylenediamine, disodium coconut oil fatty acid acyl-N-carboxymethoxyethyl-N-carboxymethyl ethylenediamine, sodium laurylaminopropionate, sodium laurylamino dipropionate, triethanolamine laurylamino propionate, sodium palm oil fatty acid acyl-N-carboxyethyl-N-hydroxyethyl ethylenediamine, betaine lauryldimethylamino acetate, betaine coconut oil alkyldimethylamino acetate, betaine stearyl dimethylamino acetate, sodium stearyldimethyl betaine, coconut oil fatty acid amidopropyl betaine, palm oil fatty acid amidopropyl betaine, amidopropyl acetate betaine laurate, amidopropyl betaine ricinoleate, stearyl dihydroxyethyl betaine and lauryl hydroxysulfobetaine;

natural surfactants such as saponin, lecithin, soybean phospholipid, hydrogenated soybean phospholipid, soybean lysophospholipid, hydrogenated soybean lysophospholipid, yolk lecithin, hydrogenated yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosyl erythritol lipid;

ultraviolet ray absorbers such as: para-aminobenzoic acid derivatives such as para-aminobenzoic acid, ethyl para-aminobenzoate, glyceryl para-aminobenzoate, amyl para-dimethyl aminobenzoate and 2-ethylhexyl para-dimethyl aminobenzoate; cinnamic acid derivatives such as benzyl cinnamate, mono-2-ethyl hexanoate glyceryl dipara-methoxycinnamate, methyl 2,4-diisopropyl cinnamate, ethyl 2,4-diisopropyl cinnamate, potassium para-methoxycinnamate, sodium para-methoxycinnamate, isopropyl para-methoxycinnamate, 2-ethylhexyl para-methoxycinnamate, 2-ethoxyethyl para-methoxycinnamate and ethyl para-ethoxycinnamate; urocanic acid derivatives such as urocanic acid and ethyl urocanate; benzophenone derivatives such as 2,4-dihydroxybenzophenone, 2,2′,4,4′-tetrahydroxybenzophenone, sodium 2-hydroxy-4-methoxy-5-sulfobenzophenone, 2-hydroxy-4-methoxybenzophenone-5-sulfonate, 2-hydroxy-4-methoxybenzophenone, 2,2′-dihydroxy-4,4′-dimethoxybenzophenone and sodium 2,2′-dihydroxy-4,4′-dimethoxy-5-sulfobenzophenone; salicylic acid derivatives such as ethylene glycol salicylate, salicylate-2-ethylhexyl, phenyl salicylate, benzyl salicylate, p-tert-butylphenyl salicylate, homomethyl salicylate and salicylate-3,3,5-trimethylcyclohexyl; 2-(2′-hydroxy-5′-methoxyphenyl)benzotriazole and 4-tert-butyl-4′-methoxybenzoyl methane;

powders and color materials such as: kaolin, silicic anhydride, magnesium aluminum silicate, sericite, talc, boron nitride, mica, montmorillonite, hemp cellulose powder, wheat starch, silk powder, maize starch; natural dyes such as nitro dyes, azo dyes, nitroso dyes, triphenylmethane dyes, xanthene dyes, quinoline dyes, anthraquinone dyes, indigo dyes, pyrene dyes, phthalocyanine dyes, flavonoid, quinone, porphyrin, water soluble annatto, sepia powder, caramel, guaiazulene, gardenia blue, gardenia yellow, cochineal, shikonin, sodium copper chlorophyllin, paprika dye, safflower red, safflower yellow, laccaic acid and riboflavin butyrate ester; carbon black, yellow iron oxide, black iron oxide, red iron oxide, iron blue, ultramarine blue, zinc oxide, chromium oxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment.

plant extracts such as Angelica keiskei extract, Uncaria gambir extract, avocado extract, sweet hydrangea leaf extract, Gynostemma pentaphyllum makino extract, Althaea officinalis extract, Arnica montana extract, oil soluble Arnica montana extract, almond extract, aloe extract, Japanese styrax benzoin extract, Ginkgo biloba extract, Stinging nettle extract, Orris rhizome root extract, fennel extract, turmeric extract, dog rose fruit extract, Echinacea leaf extract, Scutellaria root extract, Phellodendron bark extract, Japanese captis extract, barley extract, okra extract, Hypericum perforatum extract, oil soluble Hypericum perforatum extract, Lamium album extract, oil soluble Lamium album extract, Ononis spinosa root extract, Nasturtium officinale extract, orange extract, orange flower water, seaweed extract, persimmon tannin, pueraria root extract, Japanese valerian extract, cattail extract, Chamomile (matricaria) extract, oil soluble Chamomile (matricaria) extract, Chamomile (matricaria) distillate, Avena sativa (oat) kernel extract, carrot extract, oil soluble carrot extract, carrot oil, Artemisia capillaris extract, Glycyrrhiza glabra (licorice) extract, powdered Glycyrrhiza glabra (licorice) extract, Glycyrrhiza glabra (licorice) extract flavonoid, cantharides tincture, raspberry extract, kiwi extract, cinchona extract, cucumber extract, apricot kernel extract, quince seed extract, gardenia florida extract, Sasa albomarginata extract, Sophora root extract, walnut shell extract, Citrus paradisi (grapefruit) extract, Clematis vitalba leaf extract, black sugar extract, chlorella extract, mulberry bark extract, Cinnamon bark extract, Gentian extract, Geranium herb extract, black tea extract, Nuphar extract, burdock root extract, oil soluble burdock root extract, wheat germ extract, hydrolyzed wheat powder, rice bran extract, fermented rice bran extract, Symphytum officinale (comfrey) extract, Asiasarum root extract, Crocus sativus (saffron) extract, Saponaria officinalis extract, oil soluble salvia extract, Crataegus cuneata fruit extract, Zanthoxylum fruit extract, Lentinus edodes extract, powdered Lentinus edodes extract, Rehmannia root extract, Lithospermum root extract, oil soluble Lithospermum root extract, Perilla herb extract, linden extract, oil soluble Tilia europaea extract, Filipendula extract, Peony root extract, Coix lacryma-jobi extract, ginger extract, oil soluble ginger extract, ginger tincture, Acorus calamus root extract, Betula pendula (birch) extract, oil soluble Betula alba (birch) extract, Betula pendula (birch) sap, Lonicera japonica extract, Equisetum arvense extract, oil soluble Equisetum arvense extract, scordinin, stevia extract, ivy extract, Crataegus oxyacantaha (whitethorn) extract, sambucus extract, Juniperus communis extract, Achillea milefolium extract, oil soluble Achillea milefolium extract, Mentha piperita (peppermint) extract, Salvia officinalis (sage) extract, oil soluble Salvia officinalis (sage) extract, Salvia officinalis (sage) water, Malva Sylvestris (mallow) extract, Apium graveolens (celery) extract, Cnidium officinale extract, Cnidium officinale water, Swertia herb extract, Glycine max (soybean) extract, Jujube extract, thyme extract, green tea extract, tea leaf dry distilled solution, tea seed extract, clove extract, Citrus unshiu peel extract, Camellia japonica extract, Centella asiatica extract, oil soluble walnut extract, duku extract, Terminalia sericea extract, Capsicum tincture, Japanese angelica root extract, oil soluble Japanese angelica root extract, Japanese angelica root water, Calendula officinalis flower extract, oil soluble Calendula officinalis flower extract, soy milk powder, peach seed extract, Bitter orange peel extract, Houttuynia cordata extract, Solanum lycopersicum (tomato) extract, Potentilla tormentilla Schrk (Rosaceae) extract, fermented soybeans extract, Ginseng extract, oil soluble Ginseng extract, Allium sativum (garlic) extract, wild rose extract, oil soluble wild rose extract, malt extract, malt root extract, ophiopogon tuber extract, parsley extract, rye leaf juice concentrate, peppermint distillate, witch hazel distillate, witch hazel extract, rose extract, parietaria extract, Isodonis japonicus extract, Eriobotrya japonica leaf extract, oil soluble Eriobotrya japonica leaf extract, coltsfoot extract, hoelen extract, Ruscus aculeatus root extract, powdered Ruscus aculeatus root extract, grape extract, grape leaf extract, grape water, Hayflower extract, Luffa cylindrica fruit extract, Luffa cylindrica fruit water, Carthamus tinctorius (safflower) extract, oil soluble Tilia platyphyllos extract, linden distillate, Paeonia suffruticosa (peony) extract, Humulus lupulus (hops) extract, oil soluble Humulus lupulus (hops) extract, pine extract, Silybum marianum (milk thistle) extract, Aesculus hippocastanum (horse chestnut) extract, oil soluble Aesculus hippocastanum (horse chestnut) extract, Sapindus mukurossi extract, Melissa officinalis (balm mint) extract, Melilotus officinalis (melilot) extract, Prunus persica (peach) leaf extract, oil soluble Prunus persica (peach) leaf extract, bean sprouts extract, Centaurea cyanus flower extract, Centaurea cyanus flower distillate, Eucalyptus globulus extract, Saxifrage extract, Lilium (lily) extract, Coix seed extract, oil soluble Coix seed extract, Artemisia princeps pampanini extract, Artemisia princeps pampanini water, Lavandula angustifolia (lavender) extract, Lavandula angustifolia (lavender) water, apple extract, Ganoderma lucidum extract, Lactuca sativa (lettuce) extract, lemon extract, Astragalus sinicus extract, Rosa centifolia (rose) flower water, Rosemarinus officinalis (rosemary) extract, oil soluble Rosemarinus officinalis (rosemary) extract, Anthemis nobilis extract and Sanguisorba officinalis extract;

amino acids and peptides such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, DL-pyrrolidonecarboxylic acid, ε-aminocaproic acid, hydrolyzed elastin, water soluble elastin, hydrolyzed collagen, water soluble collagen, casein, glutathione, wheat peptides and soybean peptide;

vitamins and factors acting like a vitamin such as: vitamin A and analogues thereof such as retinol, retinal, retinoic acid, retinol acetate and retinol palmitate; carotenoids such as α-carotene, β-carotene, γ-carotene, δ-carotene, lycopene, zeaxanthin, cryptoxanthin, echinenon and astaxanthin; vitamin B₁ and analogues thereof such as thiamines; vitamin B₂ and analogues thereof such as riboflavin; vitamin B₆ and analogues thereof such as pyridoxine, pyridoxal and pyridoxamine; vitamin B₁₂ and analogues thereof such as cyanocobalamin; folic acids, nicotinic acid, nicotinamide, pantothenic acids, biotins; vitamin C and analogues thereof such as L-ascorbic acid, sodium L-ascorbate, L-ascorbyl stearate, L-ascorbyl palmitate, L-ascorbyl dipalmitate, L-ascorbyl tetraisopalmitate, L-ascorbate sulfate disodium ester, magnesium L-ascorbyl, sodium L-ascorbyl phosphate and L-ascorbate-2-glucoside; vitamin D and analogues thereof such as ergocalciferol and cholecalciferol; vitamin E and analogues thereof such as d-α-tocopherol, DL-α-tocopherol, dl-α-tocopherol acetate, dl-α-tocopherol succinate, β-tocopherol, γ-tocopherol and d-δ-tocopherol; ubiquinones, vitamin K and analogues thereof, carnitine, ferulic acid, γ-oryzanol, α-lipoic acid and orotic acid;

antiseptic agents such as benzoic acid, sodium benzoate, undecylenic acid, salicylic acid, sorbic acid, potassium sorbate, dehydroacetic acid, sodium dehydroacetate, isobutyl parahydroxybenzoate, isopropyl parahydroxybenzoate, ethyl parahydroxybenzoate, butyl parahydroxybenzoate, propyl parahydroxybenzoate, benzyl parahydroxybenzoate, methyl parahydroxybenzoate, sodium parahydroxybenzoate methyl, phenoxyethanol, light sensitive dye No. 101, light sensitive dye No. 201 and light sensitive dye No. 401;

antioxidizing agents such as butylhydroxyanisole, butylhydroxytoluene, propyl gallate, erythorbic acid, sodium erythorbate, para-hydroxyanisole and octyl gallate;

chelating agents to bind to a metal ion such as trisodium ethylenediamine hydroxyethyl triacetate, edetic acid, disodium edetate, trisodium edetate, tetrasodium edetate, sodium citrate, gluconic acid, phytic acid, sodium polyphosphate and sodium metaphosphate;

moisturizing agents such as hyaluronic acid, sodium hyaluronate, sodium chondroitin sulfate, sodium lactate, sodium pyrrolidone carboxylate, betaine, lactic acid bacteria fermented solution, yeast extract and ceramide;

anti-inflammatory agents such as glycyrrhizic acid, trisodium glycyrrhizinate, dipotassium glycyrrhizinate, monoammonium glycyrrhizinate, β-glycyrrhetinic acid, glycerin glycyrrhetinate, stearyl glycyrrhetinate, lysozyme chloride, hydrocortisone and allantoin;

pH adjusting agents such as sodium hydroxide, potassium hydroxide and triethanolamine;

salts such as sodium chloride, potassium chloride, magnesium chloride and sodium sulfate; α-hydroxy acids such as citric acid, glycolic acid, tartaric acid and lactic acid;

whitening agents such as arbutin, α-arbutin and placenta extract;

essential oils such as Archangelica officinalis (angelica) oil, Canangium odoratum (ylang ylang) oil, Canarium luzonicum (elemi) oil, orange oil, Chamomilla recutita (matricaria) oil, Anthemis nobilis oil, Elettaria cardamom (cardamon) oil, Acorus calamus (calamus) oil, Ferula galbaniflua (galbanum) oil, Cinnamomum camphora (camphor) oil, Daucus carota (carrot) seed oil, Salvia sclarea (clary sage) oil, Citrus paradisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedarwood) oil, Cympogon nardus (citronella) oil, Cinnamomum zeylanicum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium grabeolens (geranium) oil, Thymus vulgaris (thyme) oil, Melaleuca alternifolia (tea tree) oil, Myristica fragrans (nutmeg) oil, Melaleuca qui.viridiflara (niaouli) oil, Citrus aurantium (neroli) oil, pine oil, Ocimum basilicum (basil) oil, Mentha arvensis oil, Pogostemon patchouli (patchouli) oil, Cymbopogon martini (palmarosa) oil, Foeniculum vulgare (fennel) oil, Citrus bigaradia (petitgrain) oil, Piper nigrum (blackpepper) oil, Boswellia carterii (frankincense) oil, Vetiveria zizanoides (vetivert) oil, Mentha piperita (peppermint) oil, Citrus bergamia (bergamot) oil, benzoin oil, Aniba rosaeodora (bois de rose) oil, origanum majorana (marjoram) oil, mandarin oil, Conumiphora myrrha (myrrh) oil, Melissa officinalis (balm mint) oil, Eucalyptus globulus oil, Citrus junos oil, Citrus aurantifolia (lime) oil, Ravensara aromaticum (ravensara) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosemarinus officinalis (rosemary) oil and Levisticum officinale (lovage) oil;

terpenes such as limonene, pinene, terpinene, terpinolene, myrcene and longifeelene;

fragrance, and the like.

Furthermore, to the cosmetic of the invention may also be added any existing raw material of cosmetics at a general concentration. All raw materials of cosmetics described in, for example, Keshouhin genryou kizyun (Standards of raw materials of cosmetics), second edition, notes, edited by Society of Japanese Pharmacopoeia, 1984 (YAKUJI NIPPO LIMITED.), Keshouhin genryou kizyun-gai seibun kikaku (Standards of raw materials of cosmetics, nonstandard ingredients), under the editorship of Pharmaceutical Affairs Bureau Evaluation and Registration Division, 1993 (YAKUJI NIPPO LIMITED.), Keshouhin genryou kizyun-gai seibun kikaku tsuiho (Standards of raw materials of cosmetics, nonstandard ingredient Supplement), under the editorship of Pharmaceutical Affairs Bureau Evaluation and Registration Division, 1993 (YAKUJI NIPPO LIMITED.), Keshouhin syubetsu kyoka kizyun (Standards of cosmetic classification permission), under the editorship of Pharmaceutical Affairs Bureau Evaluation and Registration Division, 1993 (YAKUJI NIPPO LIMITED.), Keshouhin syubetsu haigou seibun kikaku (Standards of cosmetic classification ingredients), under the editorship of Pharmaceutical Affairs Bureau Evaluation and Registration Division, 1997 (YAKUJI NIPPO LIMITED.), Keshouhin genryou jiten (Dictionary of raw materials of cosmetics), 1991 (Nikko Chemicals Co., Ltd.) and the like may be used.

The oil-in-water emulsified composition of the present invention can be prepared by a general emulsification method. That is, the composition can be prepared by using general-purpose stirrers or emulsifiers such as a colloid mill, a homomixer, a high-pressure homogenizer, an ultrasonic emulsifier and the like.

The oil-in-water emulsified composition of the invention thus obtained can be suitably used in external preparations for skin such as emulsion and cream and in cosmetics for basic skin care, makeup and body care such as milky lotion, essence, skin cream, makeup base lotion, makeup base cream, milky-liquid type foundation, cream-type foundation, creamy eye color, creamy cheek color and pack.

### BEST MODE FOR CARRYING OUT THE INVENTION

The present invention is explained in more detail below by way of Examples, however the invention is by no means limited to these Examples. In Examples demonstrated below, glycerin for use had a concentration of 98 mass % or more. Sodium surfactin for use was Aminofect (registered trademark) manufactured by SHOWA DENKO K.K. What the mark “%” indicates is percentage by mass.

### EXAMPLES 1 TO 5 AND Comparative Examples 1 to 4

Emulsified compositions each having a composition as shown in Table 1 were prepared according to the preparation method described below. Using the compositions, storage stability tests were conducted. In the tests, after leaving test samples to stand at 40° C. for 8 weeks in a glass bottle, the condition and appearance were observed. Samples in which separation was observed were evaluated “x” while samples in which no separation was found were evaluated as “O”.

TABLE 1

| | | | | | | | Comp. | Comp. | Comp. | Comp. |
|---|---|---|---|---|---|---|---|---|---|---|
| composition | | Ex. 1 | Ex. 2 | Ex. 3 | Ex. 4 | Ex. 5 | Ex. 1 | Ex. 2 | Ex. 3 | Ex. 4 |
| I | sodium surfactin | 2% | 2% | 2% | 2% | 2% | 2% | — | 2% | 0.01% |
| | glycerin | 8% | 8% | 4% | 4% | 8% | 8% | 8% | 8% | 8% |
| | purified water | balance | balance | balance | balance | balance | balance | balance | balance | balance |
| II | squalane | 9.65% | 9.65% | 16% | 16% | 9.25% | 9.65% | 9.65% | 9.65% | 9.65% |
| | liquid paraffin | 16% | 16% | 11.65% | 9.65% | 16% | 16% | 16% | 16% | 16% |
| | isononyl isononanoate | 8% | 8% | 8% | 8% | 8% | 8% | 8% | 8% | 8% |
| | glyceryl tri-2-ethylhexanoate | 8% | 8% | 8% | 8% | 8% | 8% | 8% | 8% | 8% |
| | dimethicone | 8% | 8% | 6% | 8% | 8% | 8% | 8% | 8% | 8% |
| | behenyl alcohol | — | — | 4% | 4% | — | — | — | — | — |
| | cholesterol | — | — | — | — | 0.4% | — | — | — | — |
| | methylparaben | 0.1% | 0.1% | 0.1% | 0.1% | 0.1% | 0.1% | 0.1% | 0.1% | 0.1% |
| | propylparaben | 0.05% | 0.05% | 0.05% | 0.05% | 0.05% | 0.05% | 0.05% | 0.05% | 0.05% |
| III | xanthan gum | 10% | 4% | 8% | 8% | 10% | — | 10% | 0.5% | — |
| | (2% aqueous solution) | | | | | | | | | |

| | | | | | | | | | |
|---|---|---|---|---|---|---|---|---|---|
| storage stability | ◯ | ◯ | ◯ | ◯ | ◯ | X | X | X | X |

### [Preparation Method of Cosmetic]

The components in (I) and the components in (II) were separately mixed and heated to 85° C. To the mixture of the components in (I), the mixture of the components in (II) was gradually added while stirring the mixed components (I) by a homomixer, and further, the components in (III) were added thereinto, and the resultant mixture was further stirred. After cooling the mixture to 30° C. while stirring, the stirring was stopped and the resultant mixture was left standing at room temperature.

As is clear from Table 1, the emulsified compositions of the present invention (Examples 1 to 5) exhibited more excellent storage stability, in comparison with the emulsified compositions of Comparative Examples 1 to 4.

### EXAMPLE 6 AND COMPARATIVE Example 5

Emulsified compositions each having a composition as shown in Table 2 were prepared according to the preparation method described below. Using the compositions, storage stability tests as aforementioned were conducted.

TABLE 2

| | | |
|---|---|---|
| | | Comp. |
| composition | Ex. 6 | Ex. 6 |

| | | | |
|---|---|---|---|
| I | sodium surfactin | 2% | 2% |
| | glycerin | 8% | 8% |
| | purified water | balance | balance |
| II | squalane | 9.65% | 9.65% |
| | liquid paraffin | 16% | 16% |
| | isononyl isononanoate | 8% | 8% |
| | glyceryl tri-2-ethylhexanoate | 8% | 8% |
| | dimethicone | 8% | 8% |
| | cetostearyl alcohol | 8% | 8% |
| | methylparaben | 0.1% | 0.1% |
| | propylparaben | 0.05% | 0.05% |
| | xanthan gum | 10% | — |
| | (2% aqueous solution) | | |

| | | |
|---|---|---|
| storage stability | ◯ | X |

### [Method of Preparation]

The components in (I) and the components in (II) were separately mixed and heated to 85° C. To the mixture of the components in (I), the mixture of the components in (II) was gradually added while stirring the mixed components (I) by a homomixer. After cooling the mixture to 30° C. while stirring, the stirring was stopped and the resultant mixture was left standing at room temperature.

### [Results]

As is clear from Table 2, the emulsified composition of the present invention (Example 6) exhibited more excellent storage stability, in comparison with the emulsified composition of Comparative Example 5.

### 7 and 8 and Comparative Example 6

Emulsified compositions each having a composition as shown in Table 3 were prepared according to the preparation method described below. Using the compositions, storage stability tests as aforementioned were conducted.

TABLE 3

| | | | | Comp. |
|---|---|---|---|---|
| composition | | Ex. 7 | Ex. 8 | Ex. 6 |
| I | sodium surfactin | 2% | 2% | 2% |
| | glycerin | 8% | 8% | 8% |
| | cholesterol | 0.4% | 0.4% | 0.4% |
| | squalane | 21.25% | 21.25% | 21.25% |
| | cetyl alcohol | 4% | 4% | 4% |
| | isononyl isononanoate | 8% | 8% | 8% |
| | glyceryl tri-2- | 8% | 8% | 8% |
| | ethylhexanoate | | | |
| | dimethicone | 8% | 8% | 8% |
| | xanthan gum | 0.2% | 0.2% | — |
| | methylparaben | 0.1% | 0.1% | 0.1% |
| | propylparaben | 0.05% | 0.05% | 0.05% |
| II | purified water | balance | balance | balance |
| | citric acid (10% | 0.2% | — | — |
| | aqueous solution) | | | |

| | | | |
|---|---|---|---|
| storage stability | ◯ | ◯ | X |

### [Method of Preparation]

The components in (I) and the components in (II) were separately mixed and heated to 85° C. To the mixture of the components in (I), the mixture of the components in (II) was gradually added while stirring the mixed components (I) by a homomixer. After cooling the mixture to 30° C. while stirring, the stirring was stopped and the resultant mixture was left standing at room temperature.

### [Results]

As is clear from Table 3, the emulsified compositions of the present invention (Examples 7 and 8) exhibited more excellent storage stability, in comparison with the emulsified composition of Comparative Example 6.

### EXAMPLE 9

### Moisturizing Cream

A moisturizing cream having a composition as shown in Table 4 was prepared according to the preparation method described below and storage stability test as aforementioned was conducted.

TABLE 4

| composition | | Ex. 9 |
|---|---|---|
| I | sodium surfactin | 2% |
| | glycerin | 8% |
| | 1,3-buthanediol | 2% |
| | purified water | balance |
| II | squalane | 9.65% |
| | liquid paraffin | 16% |
| | isononyl isononanoate | 8% |
| | glyceryl tri-2-ethylhexanoate | 8% |
| | dimethicone | 8% |
| | cetostearyl alcohol | 8% |
| | methylparaben | 0.1% |
| | propylparaben | 0.05% |
| | xanthan gum (2% aqueous solution) | 10% |
| | sodium hyaluronate | 8% |
| | (1% aqueous solution) | |
| | dipotassium glycyrrhizinate | 0.2% |

| | |
|---|---|
| storage stability | ◯ |

### [Method of Preparation]

The components in (I) and the components in (II) were separately mixed and heated to 85° C. To the mixture of the components in (I), the mixture of the components in (II) was gradually added while stirring the components (I) by a homomixer. After cooling the mixture to 30° C. while stirring, the stirring was stopped and the resultant mixture was left standing at room temperature.

### [Results]

The obtained cream exhibited an excellent storage stability. Moreover, the cream had an excellent moisturizing action, was non-irritant and provided smooth feeling upon use.

### EXAMPLE 10

### Emollient Lotion

An emollient lotion having a composition as shown in Table 5 was prepared according to the preparation method described below and storage stability test as aforementioned was conducted.

TABLE 5

| composition | | Ex. 10 |
|---|---|---|
| I | sodium surfactin | 2% |
| | glycerin | 8% |
| | 1,3-buthanediol | 2% |
| | purified water | balance |
| II | squalane | 9.65% |
| | cetyl 2-ethylhexanoate | 8% |
| | octyldodecyl myristate | 8% |
| | isononyl isononanoate | 8% |
| | glyceryl tri-2-ethylhexanoate | 8% |
| | macadamia nut oil | 4% |
| | cetostearyl alcohol | 2% |
| | phytosterol | 0.4% |
| | methylparaben | 0.1% |
| | propylparaben | 0.05% |
| | xanthan gum | 0.2% |
| | sodium hyaluronate | 8% |
| | (1% aqueous solution) | |

| | |
|---|---|
| storage stability | ◯ |

### [Method of Preparation]

The components in (I) and the components in (II) were separately mixed and heated to 85° C. To the mixture of the components in (I), the mixture of the components in (II) was gradually added while stirring the components (I) by a homomixer. After cooling the mixture to 30° C. while stirring, the stirring was stopped and the resultant mixture was left standing at room temperature.

### [Results]

The obtained lotion exhibited an excellent storage stability. Moreover, the lotion had an excellent emollient property, was non-irritant and provided smooth feeling upon use.

### INDUSTRIAL APPLICABILITY

According to the present invention, an oil-in-water emulsified composition which is excellent in feeling upon use, moisture retention, emollient property and stability as well as environmental suitability and safety for living organisms.

1. An oil-in-water emulsified composition, which comprises 0.1 to 5% by mass of (A) lipopeptide compound derived from a microorganism, 0.05 to 1.5% by mass of (B) xanthan gum, (C) oil component and (D) water.

2. The oil-in-water emulsified composition according to claim 1, wherein the content of the oil component is from 25 to 70% by mass.

3. The oil-in-water emulsified composition according to claim 1, wherein the water content is from 15 to 55% by mass.

4. The oil-in-water emulsified composition according to claim 1, wherein the microorganism-derived lipopeptide compound (A) is at least one species selected from surfactins, its analogous compounds and salts thereof.

5. The oil-in-water emulsified composition according to claim 4, wherein the surfactin or its analogous compound comprises at least one or more compounds as represented by the formula (1) below: 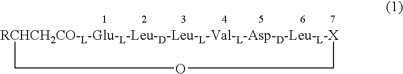
wherein X represents an amino acid residue selected from the group consisting of leucine, isoleucine, valine, glycine, serine, alanine, threonine, asparagine, glutamine, aspartic acid, glutamic acid, lysine, arginine, cysteine, methionine, phenylalanine, tyrosine, tryptophan, histidine, proline, 4-hydroxyproline and homoserine, and R represents a normal alkyl group having from 8 to 14 carbon atoms, an isoalkyl group having from 8 to 14 carbon atoms or an anteisoalkyl group having from 8 to 14 carbon atoms.

6. The oil-in-water emulsified composition according to claim 5, wherein X is residue of leucine, isoleucine or valine.

7. The oil-in-water emulsified composition according to claim 4, wherein the salt is at least one compound selected from the group consisting of sodium salt, potassium salt, monoethanolamine salt, diethanolamine salt, triethanolamine salt, arginine salt and lysine salt.

8. The oil-in-water emulsified composition according to claim 4, wherein the microorganism-derived lipopeptide compound (A) is sodium surfactin.

9. The oil-in-water emulsified composition according to claim 1, comprising no nonionic surfactant.

10. The oil-in-water emulsified composition according to claim 1, comprising no acrylic acid-based water-soluble polymer.

11. An external preparation for skin comprising the oil-in-water emulsified composition according to claim 1.

12. A cosmetic comprising the oil-in-water emulsified composition according to claim 1.
